(19) United States
(12) Patent Application Publication
(10) Pub. No.: US 2021/0335122 A1
(43) Pub. Date: Oct. 28, 2021

(54) APPARATUS AND METHOD FOR PROMOTING HAND HYGIENE

(71) Applicant: MURAD MAHMOUD, København Ø (DK)

(72) Inventor: MURAD MAHMOUD, Copenhagen (DK)

(21) Appl. No.: 17/354,420

(22) Filed: Jun. 22, 2021

Related U.S. Application Data
(60) Provisional application No. 63/013,580, filed on Apr. 22, 2020.

(51) Int. Cl. G08B 21/24; G16H 40/20; G08B 5/36; G08B 3/10; A47K 5/12

(52) U.S. Cl. CPC G08B 21/245; G16H 40/20; A47K 5/1217; G08B 3/10; G08B 5/36

(57) ABSTRACT

An apparatus for promoting effective handwashing is disclosed. The apparatus comprises a body removably connected to a dispenser. The body houses at least one microcontroller, a speaker, a timer, two or more light-emitting diodes (LEDs), and a plurality of sensors. The apparatus is configured to detect the presence of a user within the range of the apparatus using a PIR sensor and a microphone. Further, the apparatus nudges the user to wash their hands by generating a first audio-visual alert. A timer is initiated upon determining that the user has begun washing their hands. Further, a second audio-visual alert is generated for notifying improper hand wash if the user leaves the handwashing area before the timer completes the target runtime. A third audio-visual alert is generated for notifying proper hand wash if the user stays within the handwashing area until the timer completes the target runtime.

### CROSS-REFERENCE TO RELATED APPLICATIONS

This application claims the benefit of U.S. Provisional Patent Application Ser. No. 63/013,580 entitled, “TECHNIQUES FOR PROMOTING AND MAINTAINING HAND HYGIENE” filed on Apr. 22, 2020, which is incorporated herein in its entirety.

### TECHNICAL FIELD

The present invention relates generally to techniques for monitoring and promoting hand hygiene. More particularly, the present invention is related to an apparatus and a method for promoting hand hygiene among individuals.

### BACKGROUND

According to medicine and public health, communicable diseases are transmitted through various means of disease transmission. Transmission herein refers to the passing of a pathogen causing communicable disease from an infected host individual or group to a particular individual or group, regardless of whether the other individual was previously infected. Transmission may be airborne, direct via physical contact, or indirect via physical/surface contact. Transmission may also be caused via an intermediate host such as mosquitoes, pets, or other animals.

Airborne transmission may take place when an infected individual sneezes/coughs or even breathes near a person with no infection. Pathogens may travel via air as microparticles and infect healthy individuals. Small dry and wet particles that stay in the air for long periods allow airborne contamination even after the departure of the host. Droplet infection takes place when small and usually wet particles stay in the air for a short period. Contamination usually occurs in the presence of the host when it comes to droplet infection.

In case of contamination through direct physical contact, touching an infected individual may spread the disease. Contamination through indirect physical contact takes place when an infected person touches a surface and contaminates it. Such contamination occurs usually from unwashed hands, contaminated food, or water sources due to lack of proper sanitation and hygiene.

Studies show that 81% of infections are transferred by direct and indirect physical contact. Direct physical contact includes behaviors such as touching, handshakes, hugging, kissing, etc. While these behavioral patterns can be limited to an extent by individuals during epidemics/pandemics, it is very difficult to avoid transmission through indirect physical contact. In one example, a person with an infectious disease may sneeze into his/her hands and later handle documents or touch surfaces using their hands. This causes the contaminants to spread onto different surfaces. In such a scenario, a healthy individual who touches these surfaces may contact infection indirectly.

While it is well known that basic hygiene habits such as hand hygiene can prevent the spread of many such diseases, it is noted that under 50% of humans comply with hand hygiene guidelines globally. It has been noted that educational strategies are not sufficient to drive behavioral change. For example, the COVID-19 outbreak could have been controlled to an extent in many geographies and communities if basic hand hygiene was strictly followed. While organizations such as the World Health Organization (WHO) and experts around the world have repeatedly mentioned the benefits of washing hands with soap for 20 seconds for effectively removing harmful microorganisms from hands, very few individuals follow it properly. The problem with improper handwashing has been widely observed even among individuals residing in highly developed communities. Furthermore, many individuals fail to clean every part of their hands when hand wash is performed quickly making the process ineffective.

Furthermore, it has been established by numerous researchers that alcohol-based hand sanitizers are effective in killing pathogens including viruses such as the novel coronavirus (SARS-CoV-2). WHO recommends alcohol-based hand sanitizers as an effective alternative to soap for killing the virus that causes COVID-19. Based on these recommendations sanitizing check-points have been set up in many crowded areas around the world. The check-points are equipped either with facilities for washing hands with soap or with hand sanitizing stations that provide alcohol-based hand sanitizers.

The pandemic caused by the novel coronavirus has established that pathogens such as novel coronavirus can cripple the global economy and kill millions of individuals in a matter of few months. Humans have realized that the threat caused by such invisible microorganisms is far greater than any other threat ever faced by humans. A lone super-spreader can wreak havoc and bring down a whole community, geography, or country in a matter of weeks. While threats caused by humans such as terrorism, geopolitical conflicts, and the like, may be detected and controlled as they are visible to plain eyes, threats caused by such invisible killer pathogens can be overwhelming and catastrophic.

While pathogens such as bacteria may be detected using expensive machines to an extent, the possibility of these apparatuses detecting viruses that are much smaller in size compared to bacteria is not practical. Many geographical areas have established the usefulness of strictly following rudimentary hygiene and implementing basic behavioral changes (such as social distancing) to combat pandemics effectively. Hence, simple, practical, and affordable solutions that can help humans in defending such pathogens are the need of the hour.

U.S. Pat. No. 8,237,558B2 discloses a system and method of encouraging compliance of hand hygiene in an environment where users move from zone to zone and are required to perform hand hygiene between the zones. Users carry a wearable zone sensor which detects zones, detects hand hygiene actions, logs time of changing zones, and hand hygiene actions. The wearable sensor can be integral with a wearable hand hygiene product dispenser and/or can operate in cooperation with a fixed dispenser configured to transmit hand hygiene actions to the wearable zone sensor. The wearable zone sensors are configured to be useable anonymously or to be associated with a user identifier, and to interface with a central computer via a docking station or communication interface to transfer data for later analysis.

U.S. Pat. No. 8,279,063B2 discloses a wireless time-of-flight distance measurement device and/or a motion detector is used at each of a plurality of stations in a wireless network in an enclosed facility to accurately locate a badge-wearing person near the station. The location, badge number and time of detection are transmitted through the network and stored in a computer memory. In a health care facility, hand-washing detectors are located at some of the stations and caused to energize a hand-wash status indicator light on the badge when the wearer has washed his or her hands. The light remains “on” for only a certain length of time, but will be extinguished sooner by a monitor device near each patient when the health care worker leaves the vicinity of the patient. These events also are transmitted and stored so that a timed record of each worker's hand-washing and visits to patients is created.

U.S. Pat. No. 8,648,724B2 discloses a system and method for motivating or prompting persons to wash hands. The system includes a sensor for detecting use of a toilet or a urinal, which sensor creates a first signal indicative of that use, and a signaling arrangement for issuing in response to the first signal at least one second signal reminding or prompting the person to use a cleansing agent dispenser.

U.S. Ser. No. 10/977,886B2 discloses a modular people counter including a base and a communications module. The base has a people counter processor, memory, a power supply having one or more batteries, a passive sensor that detects whether a badge and/or a person has entered into a selected area and is within the sensing range and a first connector. The first connector is in circuit communication with the people counter processor. The communications module has a second connector for connecting to the first connector and wireless communications circuitry. The wireless communications module is configured to receive one or more signals from the people counter processor when the first connector is connected to the second connector.

Although the abovementioned prior-arts disclose solutions for addressing issues related to hand hygiene compliance, they lack simplicity and portability. A solution that is simple and portable that monitors user behavior and generates useful recommendations for improving hand hygiene compliance in a facility is the need of the hour.

### SUMMARY

In light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole.

Embodiments disclosed herein describe an apparatus and related methods for promoting effective handwashing. The apparatus comprises a body removably connected to a handwashing agent dispenser. The body acts as a housing for various components of the apparatus including at least one microcontroller, a speaker, a timer, two or more light-emitting diodes (LEDs), and a plurality of sensors including a PIR sensor, a motion detection sensor, an accelerometer sensor, a pressure sensor, and a microphone.

The microcontroller may be configured or programmed to detect the presence of a user within a handwashing area based on signals received from at least one of: the PIR sensor, motion sensors, and the microphone, nudge the user to wash their hands by generating a first audio alert and/or a visual alert, determine the user dispensing a cleaning agent from a dispenser based on signals received from at least one of: the accelerometer sensor and the pressure sensor, initiate the timer that counts to a target runtime upon detecting the user dispensing the cleaning agent, monitor and confirm the presence of the user within the handwashing area based on the signals received from at least one of: the PIR sensor, the motion sensor and the microphone that detects sound of running water, generate a second audio and/or visual alert for notifying improper hand wash if the user leaves the handwashing area before the timer completes the target runtime, and generate a third audio and/or visual alert for notifying proper hand wash if the user stays within the handwashing area until the timer completes the target runtime.

Embodiments of the present disclosure further proposes a method for promoting effective hand hygiene comprising the steps of: detecting the presence of a user within a handwashing area based on signals received from at least one of: a passive infrared (PIR) sensor, a motion sensor and a microphone, nudging the user to wash their hands by generating a first audio and/or visual alert, determining the user dispensing a cleaning agent from a dispenser based on signals received from at least one of: an accelerometer sensor and a pressure sensor, initiating a timer that counts to a target runtime upon detecting the user dispensing the cleaning agent, monitoring and confirming the presence of the user within the handwashing area based on signals received from at least one of: the proximity sensor, the thermal sensor and the microphone that detects sound of running water, if the user leaves the handwashing area before the timer completes the target runtime, generating a second audio and/or visual alert for notifying improper hand wash, and if the user stays within the handwashing area until the timer completes the target runtime, generating a third audio and/or visual alert for notifying proper hand wash.

This summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following detailed description and figures.

The abovementioned embodiments and further variations of the proposed invention are discussed further in the detailed description.

### BRIEF DESCRIPTION OF THE DRAWINGS

FIG. 1 is an exemplary front isometric view of the front shell of the body of the apparatus for promoting hand hygiene according to the embodiments of the present disclosure;

FIG. 2 is an exemplary rear isometric view of the front shell of the body of the apparatus for promoting hand hygiene according to the embodiments of the present disclosure;

FIG. 3 is an exemplary back lid of the body of the apparatus for promoting hand hygiene according to the embodiments of the present disclosure;

FIG. 4 is an exemplary front view supporting component provided within the body of the apparatus for promoting hand hygiene according to the embodiment of the present disclosure;

FIG. 5 is an exemplary rear view of the supporting component provided within the body of the apparatus for promoting hand hygiene according to the embodiments of the present disclosure;

FIG. 6 is an exemplary front view of the motherboard of the apparatus for promoting hand hygiene according to the embodiments of the present disclosure;

FIG. 7 is an exemplary rear view of the motherboard of the apparatus for promoting hand hygiene according to the embodiments of the present disclosure;

FIG. 8 is an exemplary view of the supporting component connected the motherboard of the apparatus according to the embodiments of the present disclosure;

FIG. 9 is an exemplary explosive view of the components of the apparatus according to the embodiment of the present disclosure;

FIG. 10 is an exemplary front view of the body of the apparatus for promoting hand hygiene according to the embodiment of the present disclosure;

FIG. 11 is an exemplary rear view of the body of the apparatus for promoting hand hygiene according to the embodiment of the present disclosure;

FIG. 12 is an exemplary side view of the body of the apparatus for promoting hand hygiene according to the embodiment of the present disclosure;

FIG. 13 is an exemplary depiction of the apparatus for promoting hand hygiene fastened to a first dispenser according to the embodiment of the present disclosure;

FIG. 14 is an exemplary depiction of the apparatus for promoting hand hygiene fastened to a second dispenser according to the embodiment of the present disclosure;

FIG. 15 is a block diagram of the architecture of the components of the apparatus for promoting hand hygiene according to the embodiments of the present disclosure;

FIG. 16 is a block diagram of an electronic device/client device connected to the apparatus for promoting hand hygiene according to the embodiments of the present disclosure;

FIG. 17 is a flow diagram depicting the method for promoting hand hygiene according to the embodiments of the present disclosure.

The drawings described herein are for illustration purposes only and are not intended to limit the scope of the present subject matter in any way.

### DETAILED DESCRIPTION

The present disclosure proposes a novel solution for promoting hand hygiene among individuals. The apparatus disclosed by the present disclosure may include a body with an outer shell that may act as housing for various components of the apparatus. The components of the apparatus may include at least one microcontroller, a speaker, a timer, two or more light-emitting diodes (LEDs), and a plurality of sensors, among other commonly known supporting electronic components. The plurality of sensors includes passive infrared (PIR) sensors, accelerometer sensors, motion detection sensors, pressure sensors, microphones, and the like.

In the following description of the embodiments of the invention, reference is made to the accompanying drawings that form a part hereof, and in which are shown by way of illustration specific embodiments in which the invention may be practiced. These embodiments are described in sufficient detail to enable those skilled in the art to practice the invention, and it is to be understood that other embodiments may be utilized and that changes may be made without departing from the scope of the present invention. The following detailed description is, therefore, not to be taken in a limited sense, and the scope of the present invention is defined only by the appended claims.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

As used herein, the singular forms “a”, “an”, and “the” are intended to include the plural forms as well, unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the foregoing sections, some features are grouped together in a single embodiment for streamlining the disclosure. This method of disclosure is not to be interpreted as reflecting an intention that the disclosed embodiments of the present disclosure must use more features than are expressly recited in each claim. Rather, as the following claims reflect, the inventive subject matter lies in less than all features of a single disclosed embodiment. Thus, the following claims are hereby incorporated into the detailed description, with each claim standing on its own as a separate embodiment.

In the present disclosure, the terms ‘washing’, ‘cleaning’, and ‘sanitizing’ may be used interchangeably. The terms ‘hand cleaning agent’, ‘sanitizer’, ‘liquid soap’, and ‘soap’ may be used interchangeably. The terms used herein do not restrict the scope of the present invention. The terms used herein cover all possible technical solutions as understood by a person skilled in the art.

The apparatus may be installed by fastening it onto a dispenser that dispenses a hand cleaning agent. The hand cleaning agent may be an alcohol-based hand sanitizing gel/solution, a liquid soap, cleaning gel, or any commercially available cleaning agent. The apparatus along with the dispenser may be installed at various locations indoors as well as outdoors. They may be installed in locations such as washrooms, entrances and exits of rooms/buildings, etc., where individuals are usually expected to clean their hands. The apparatus may also be retrofitted into existing dispensers by replacing the body of the apparatus with the body of the dispenser.

The microcontroller may be configured or programmed to detect the presence of a user near the apparatus designated as a hand cleaning area. The designated area may include a predetermined area around the apparatus as measured by the sensors provided in the apparatus. The predefined area may be referred to as the ‘range of the apparatus’. Once the apparatus is installed in a location, it may start monitoring individuals coming within the range of the apparatus using a combination of signals received from the PIR sensor, motion sensor, and the microphone. Upon determining the presence of an individual within the range of the apparatus, the apparatus nudges the user to wash their hands by generating a first audio and/or visual alert. The first audio and/or visual alert may include blinking the LEDs in a predefined pattern along with a generation of audio such as a beep sound to attract the attention of the individual towards the apparatus.

The next step performed by the apparatus includes determining the user dispensing the cleaning agent from a dispenser. This is achieved by processing the signals received from at least one of the accelerometer sensor and the pressure sensor provided within the apparatus. The sensors may detect pressure applied or a dispensing action performed on the dispenser to confirm that the user has dispensed the cleaning agent from the dispenser. Upon determining the dispensing action, the microcontroller may initiate the timer that counts to a target runtime. In one example, the target runtime maybe 20 seconds as recommended by the World Health Organization (WHO). Alternatively, the runtime may be increased to include the time required for washing off the agent from the hands of the user.

The microcontroller may simultaneously monitor and confirm the presence of the user within the range of the apparatus based on the signals received from a combination of: the PIR sensor and/or the microphone. Herein, the PIR sensor may detect proximity and moisture/temperature to detect the presence of the user in its range while the microphone detects the sound of running water from the tap to determine the presence of the user. Motion sensors may also be used instead of PIR sensors to detect the presence of the user and/or the user's hands. If the user leaves the range of the apparatus before the timer completes the target runtime (20 seconds or the preset time period), a second audio and/or visual alert for notifying improper hand wash may be generated. In one example, the second audio and/or visual alert may include audio that sounds like a warning sign along with LED lights blink in red color. Different combinations of audio and/or visual signs may be provided by the apparatus depending on the conditions set by the user. Users may be provided with options for customizing the audio and/or visual alerts. If the user remains within the range of the apparatus until the timer hits the target runtime, a third audio and/or visual alert for notifying proper hand wash may be generated.

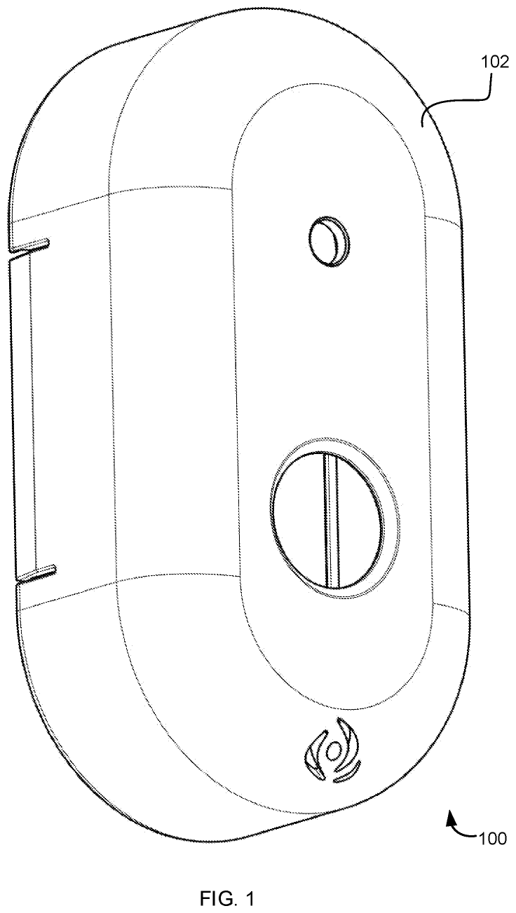

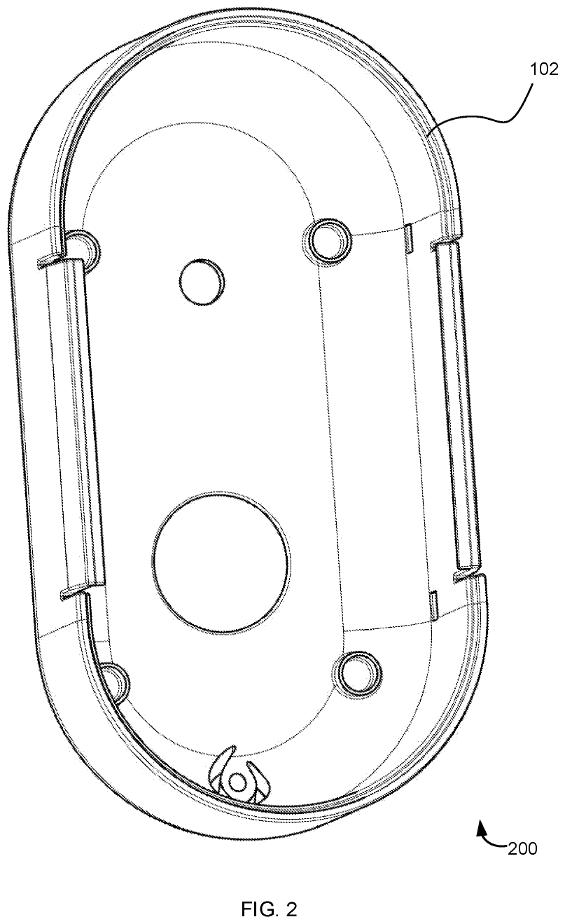

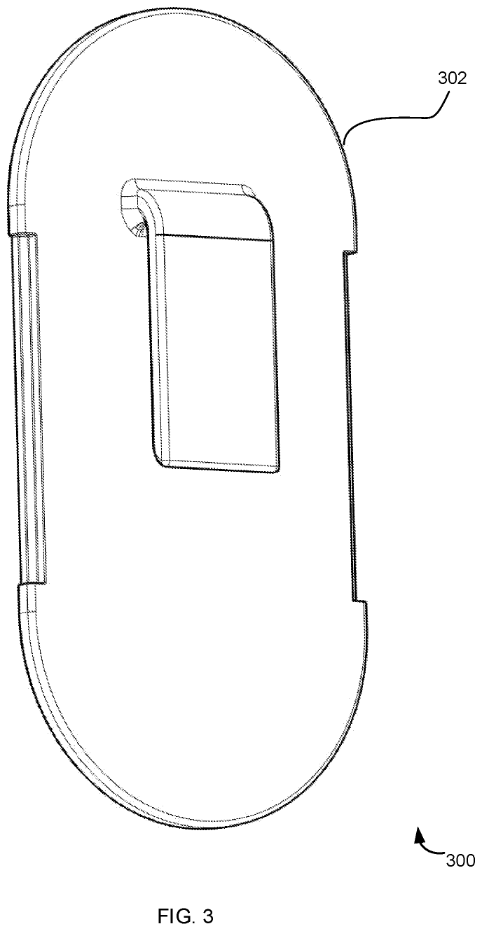

Referring to the figures, FIG. 1 is a front isometric view 100 of the front shell 102 of the body of the apparatus described herein. FIG. 2 is a rear isometric view 200 of the front shell of the body of the apparatus. The front shell may be designed in different shapes and sizes as understood by a person skilled in the art. The exemplary design provided herein does not restrict the scope of the present disclosure with respect to its ornamental design. FIG. 3 is an exemplary view 300 of the back lid 302 of the apparatus. The front shell 102 and the back lid 302 together form the outermost shell of the body of the apparatus.

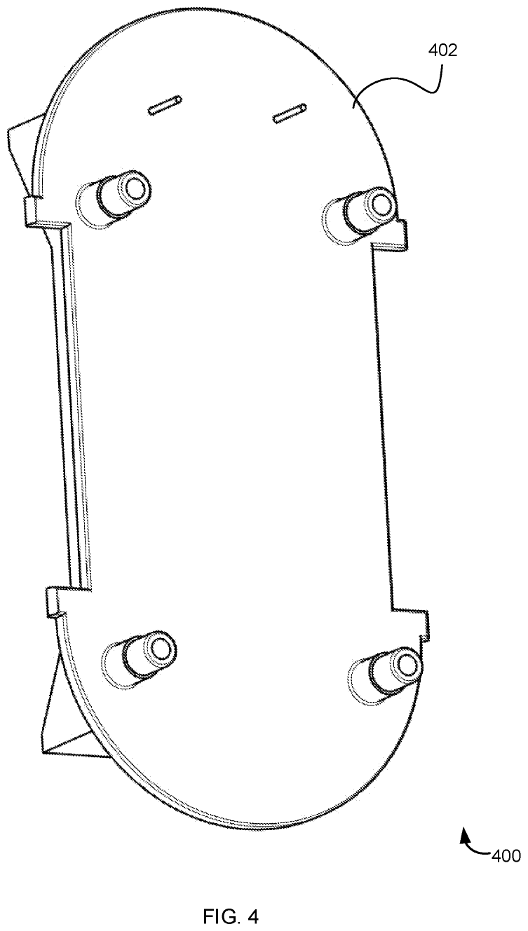

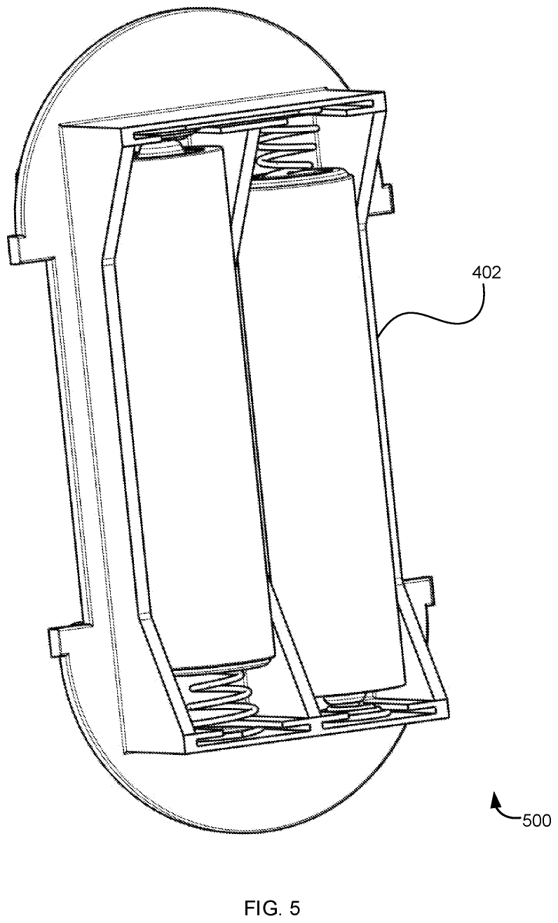

FIG. 4 is a front view 400 a supporting component 402 provided within the body of the apparatus according to the embodiment of the present disclosure. The supporting component 402 may house the power source such as a battery that powers the apparatus. The front side of the supporting component 402 may be affixed with the motherboard of the apparatus while the rear side may house the batteries. FIG. 5 is an exemplary rear view 500 of the supporting component 402 provided within the body of the apparatus according to the embodiments of the present disclosure. The rear side of the supporting component may house the batteries as described in the figure. It may be noted that the power source used may vary depending upon the use case of the apparatus. While some embodiments may use rechargeable batteries others may use replaceable batteries. In another example, instead of batteries, the apparatus may be directly plugged into a power source such as an AC/DC power source that may continuously provide the power required for running the apparatus.

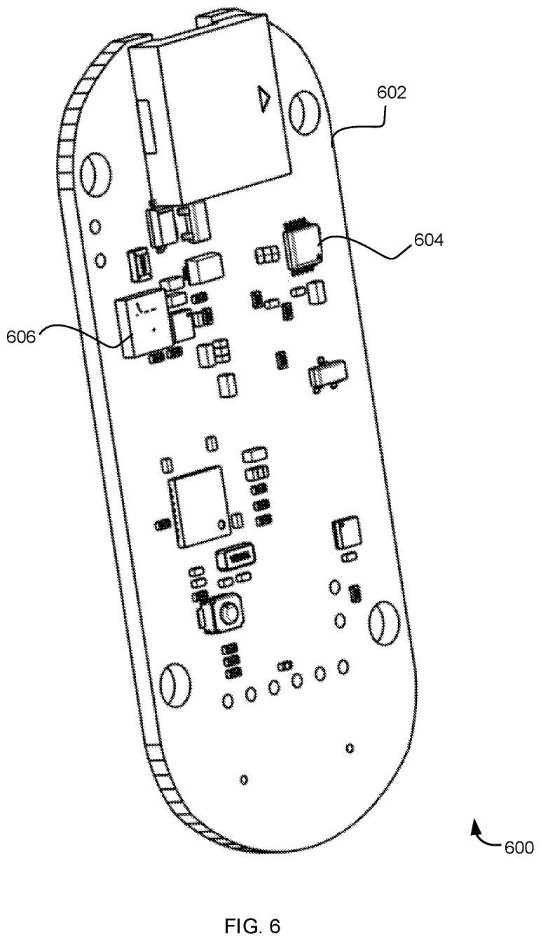

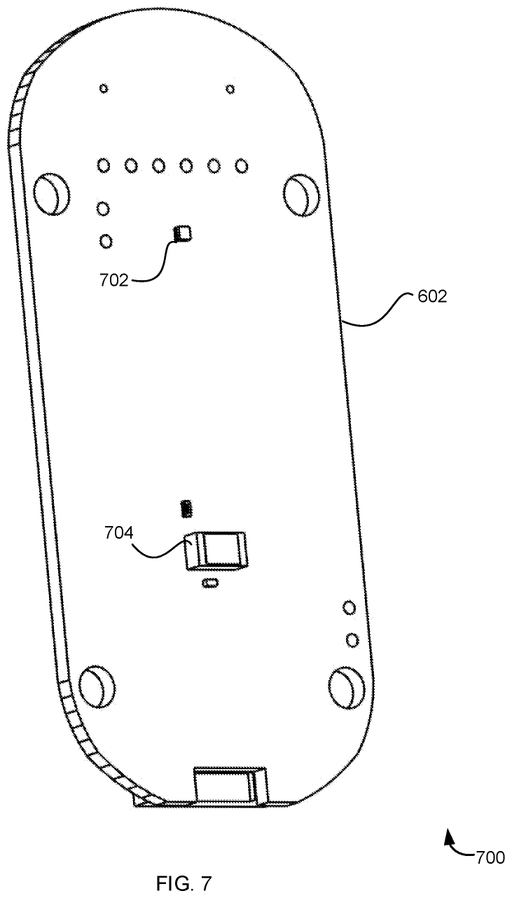

FIG. 6 is an exemplary front view 600 of the motherboard 602 of the apparatus and FIG. 7 is an exemplary rear view 700 of the motherboard 602 of the apparatus for promoting hand hygiene according to the embodiments of the present disclosure. The motherboard 602 may be a printed circuit board on which the components of the apparatus such as the microcontroller 604, the plurality of sensors, the speaker 606, the LED lights, and the like, may be mounted. The motherboard 602 may be mounted on top of the supporting component 402. The LED lights 702 and the PIR sensor 704 may slightly extend outwards from the front side of the motherboard 602.

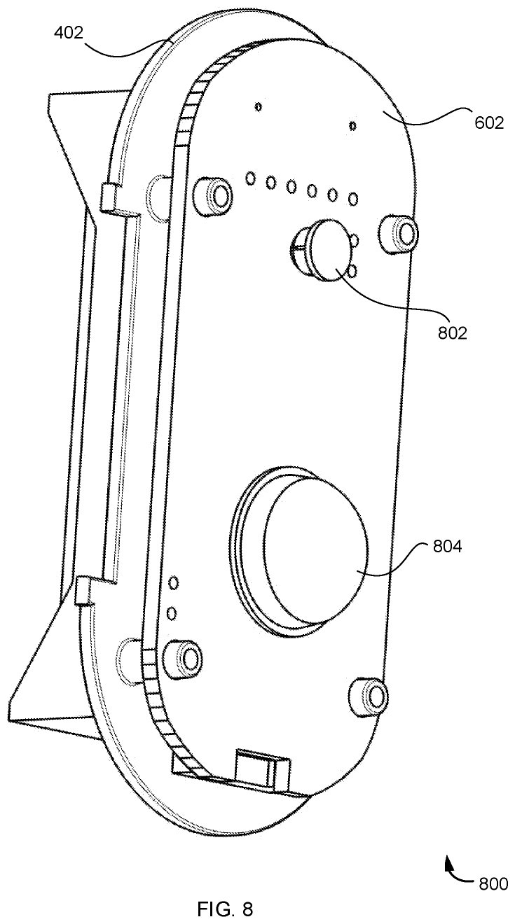

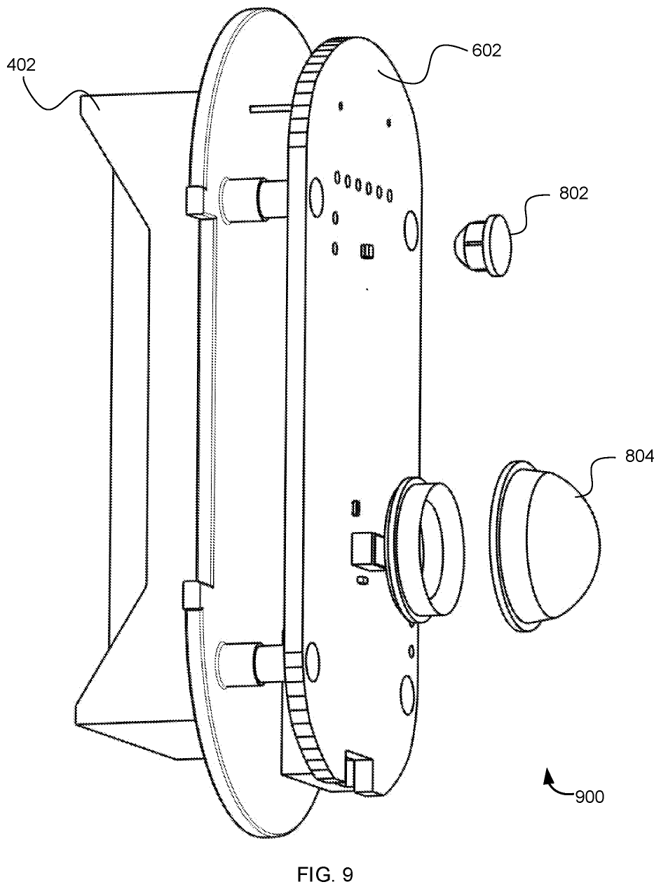

FIG. 8 is an exemplary view 800 of the supporting component 402 connected the motherboard 602 of the apparatus. A light guide 802 may be provided directly in front of the LED light 702 and a lens 804 may be provided in front of the sensor 704. The front shell 102 may be provided with provisions for the light guide 802 and the lens 804 to extend slightly outwards from the body of the apparatus for better visibility and functioning. FIG. 9 is an exemplary explosive view 900 of the component of the apparatus according to the embodiment of the present disclosure.

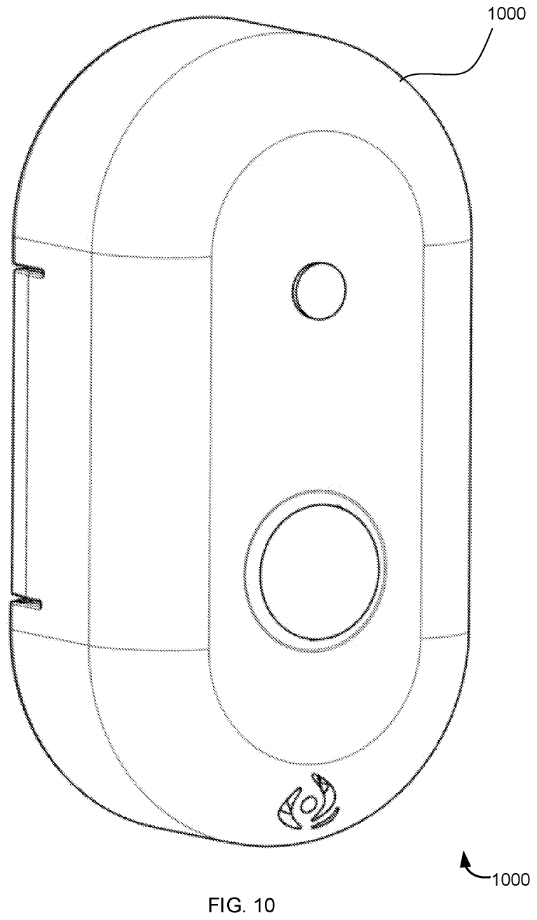

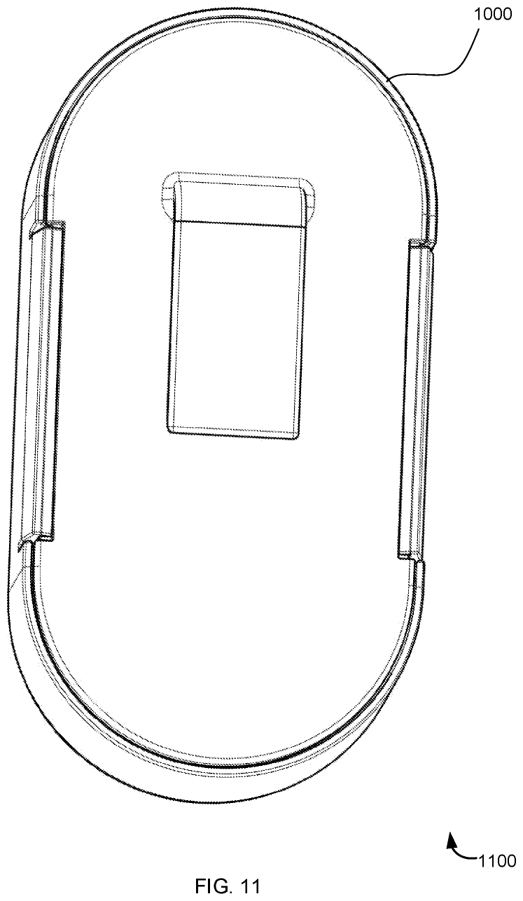

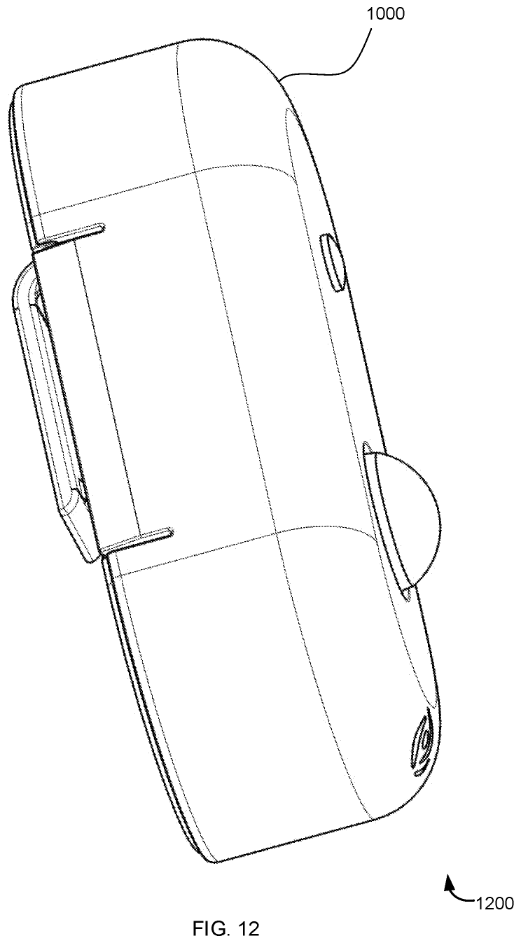

FIG. 10 is an exemplary front view 1000 of the body of the apparatus, FIG. 11 is an exemplary rear view 1100 of the body of the apparatus, and FIG. 12 is an exemplary side view 1200 of the body of the apparatus according to the embodiment of the present disclosure. It may be noted that the design of the apparatus provided in the figures are merely exemplary and does not restrict the scope of the present subject matter.

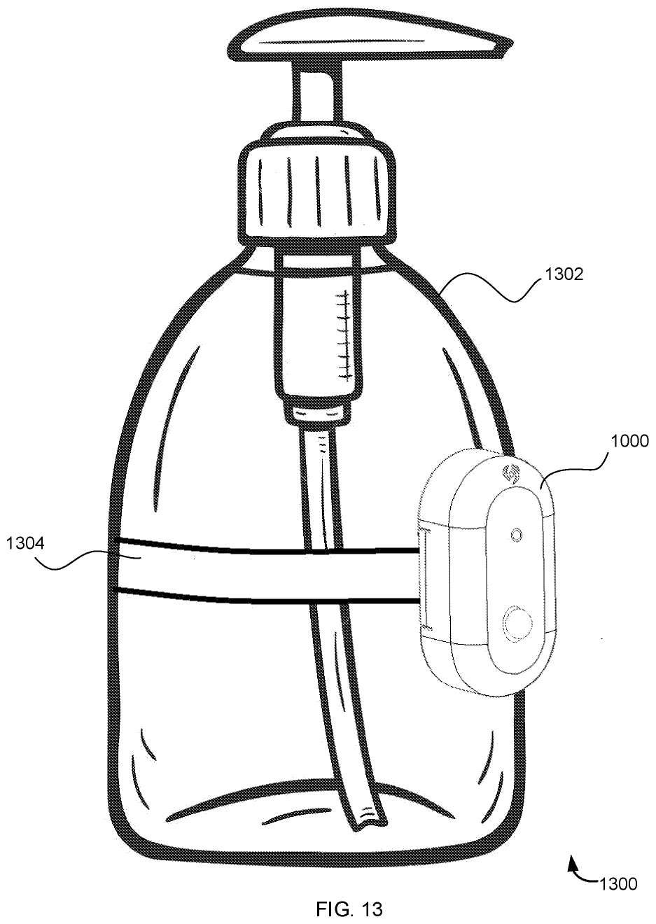

FIG. 13 is an exemplary depiction 1300 of a first use case of the apparatus for promoting hand hygiene fastened to an exemplary first dispenser. In this embodiment, the apparatus maybe fastened to a generic hand wash liquid dispenser 1302 by fastening the apparatus to the dispenser using a fastening means 1304 such as brackets, screws, adhesives, bands, vacuum suction cups, or any other well-known fastening means. The cleaning agent provided in the dispenser may be a soap-based cleaning agent, an alcohol-based hand sanitizer, or any other well-known sanitizing agent known to a person skilled in the art.

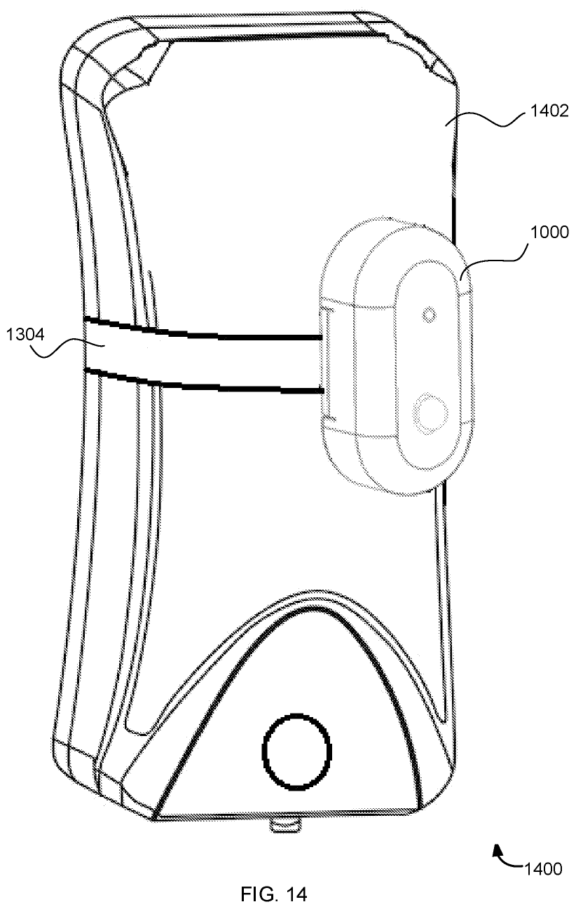

FIG. 14 is an exemplary depiction 1400 of a second use case of the apparatus for promoting hand hygiene fastened to a second dispenser 1402 according to the embodiment of the present disclosure. In this embodiment, the apparatus may be fastened to a wall-mounted hand cleaning agent dispenser. The cleaning agent provided in the dispenser may be a soap-based cleaning agent, an alcohol-based hand sanitizer, or any other well-known sanitizing agent known to a person skilled in the art. The second dispenser may automatically dispense the hand cleaning agent on the user's hands when the user's hands are placed directly below the second dispenser. While the first dispenser 1302 may dispense the hand cleaning agent upon the user applying pressure on the nozzle, the second dispenser 1402 may dispense the hand cleaning agent either by pressing a button provided on the dispenser or automatically upon detecting the user's hands below the nozzle.

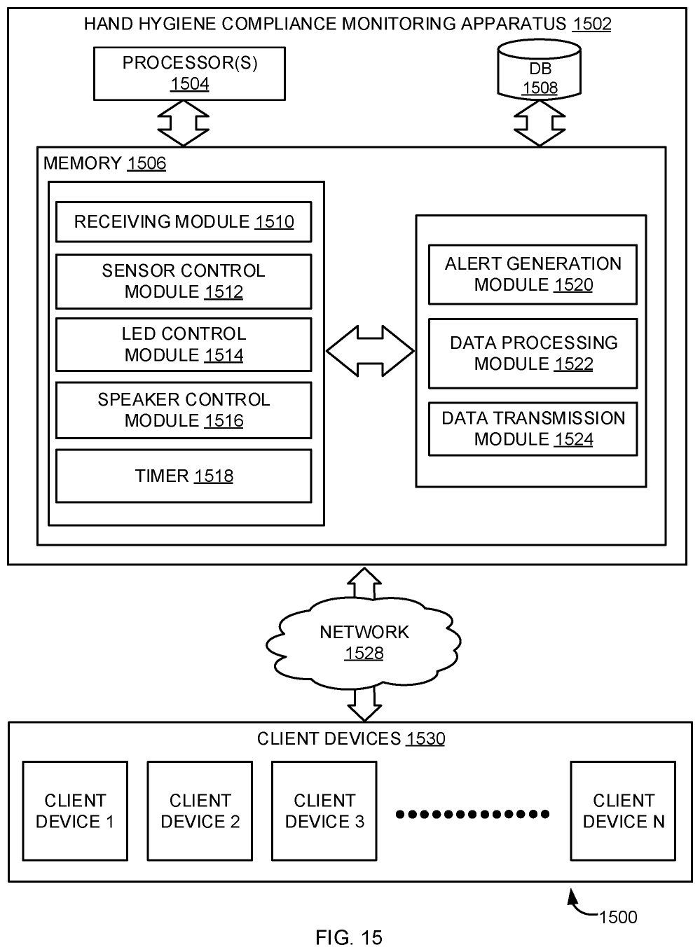

FIG. 15 is a block diagram 1500 of the architecture of a handwashing compliance monitoring system 1502 connected to a plurality of client devices 1530 via a network 1528. As shown in FIG. 15, the system 1502 may include processor(s) 1504 and memory 1506 that are communicatively coupled to each other. Further, the system 1502 may include a database (DB) 1508 that may be communicatively connected to the memory 1506. Furthermore, as shown in FIG. 15, memory 1506 may include a receiving module 1510, a sensor control module 1512, a LED control module 1514, a speaker control module 1516, a timer 1518, an alert generation module 1520, a data processing module 1522, and a data transmission module 1524. One or more client devices 130 may be communicatively connected to the hand hygiene compliance monitoring system 1502.

In one example, the microcontroller 604 of the apparatus may host the processor 1504 as well as the memory 1506. Alternatively, the processor 1504 and the memory 1506 may be provided separately. During operation, the modules described above may be loaded into the memory 1506 and may be processed by the processor 1504.

Components of the hand hygiene compliance monitoring system 1502 may be any combination of hardware and programming to implement the functionalities described herein. In some implementations, the programming may be processor 1504 executable instructions stored on a non-transitory machine-readable storage medium (e.g., memory 1506), and the hardware may include at least one processing resource to retrieve and/or execute those instructions. Processor(s) 1504 may include, but are not limited to, one or more digital signal processors (DSPs), one or more microprocessor, one or more special-purpose computer chips, one or more field-programmable gate arrays (FPGAs), one or more application-specific integrated circuits (ASICs), one or more computer(s), various analog to digital converters, digital to analog converters, and/or other support circuits. Processor(s) 1504 thus may also include the functionality to encode messages and/or data or information. Processor(s) 1504 may include, among other things, a clock, an arithmetic logic unit (ALU), and logic gates configured to support the operation of processor(s) 1504. Further, the processor(s) 1504 may include functionality to execute one or more software programs, which may be stored in the memory 106 or otherwise accessible to processor(s) 1504.

Memory 1506, may store any number of pieces of information, and data, used by the system to implement the functions of the system. The memory 1506 may include, for example, volatile memory and/or non-volatile memory. Examples of volatile memory may include but are not limited to volatile random-access memory (RAM). The non-volatile memory may additionally or alternatively comprise an electrically erasable programmable read-only memory (EEPROM), flash memory, hard drive, and the like. Some examples of volatile memory include, but are not limited to, dynamic RAM, static RAM, and the like. Some example of the non-volatile memory includes, but are not limited to, hard disks, magnetic tapes, optical disks, programmable read-only memory, erasable programmable read-only memory, electrically erasable programmable read-only memory, flash memory, and the like. Memory 106 may be configured to store information, data, applications, instructions, or the like for enabling the system to carry out various functions in accordance with various example embodiments. Additionally, or alternatively, the memory 1506 may be configured to store instructions which when executed by processor(s) 1504 cause the hand hygiene compliance monitoring system 1502 to behave in a manner as described in various embodiments.

In one implementation, the network 1528 may be a wireless network, a wired network, or a combination thereof. Network 1528 may be implemented as one of the several types of networks, such as intranet, local area network (LAN), wide area network (WAN), the internet, and the like. Network 1528 may either be a dedicated network or a shared network. The shared network represents an association of the several types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 1528 may include a variety of network devices, including routers, bridges, servers, computing devices, storage devices, and the like.

During the operation, the receiving module 1510 may receive data from the plurality of sensors managed by the sensor control module 1512. The LED control module changes the color and frequency of the LEDs 702 depending on the signal provided. In one example, the LEDs 702 may blink in blue color to attract the attention of the user when the user enters the range of the apparatus. The LEDs 702 may blink in a different color when the timer is running. The LEDs 702 may blink in red color if an improper hand wash is detected. The LEDs 702 may blink in green color if a proper hand wash is recorded. The colors described above are merely exemplary in nature. The lights may be configured to blink or to stay awake depending upon the use case. The lights described herein are not restricted to LEDs. Other well-known types of lights may also be used by the apparatus.

While the lights provide visual cues, the speaker 606 may provide audio alerts. The speaker may be configured to provide a wide variety of tones depending upon the use case. The speaker may be configured to make beep sounds of different frequencies or may be configured to output voice-based audio to make it more effective. Since individuals may respond better to human voice than tones, the former may be used in some cases as audio cues. Audio messages such as “please clean your hands before entering/exiting”, “well done”, “improper hand wash detected”, “timer activated”, and the like may be played to improve the efficiency of handwashing among the users. The speaker control module 1516 may be configured to perform the abovementioned functions. The timer 1518 may count the amount of time each hand wash session lasts and transfers the data to the data processing module 1522. The alert generation module 1520 generates alerts and sends them to the LED control module 1514 and the speaker control module 1516. The data processing module 1522 is configured to process the data received by the receiving module 1510 and send instructions to the alert generation module 1520. The data transmission module 1524 is configured to transmit processed data to external devices such as client devices 1530 via network 1528.

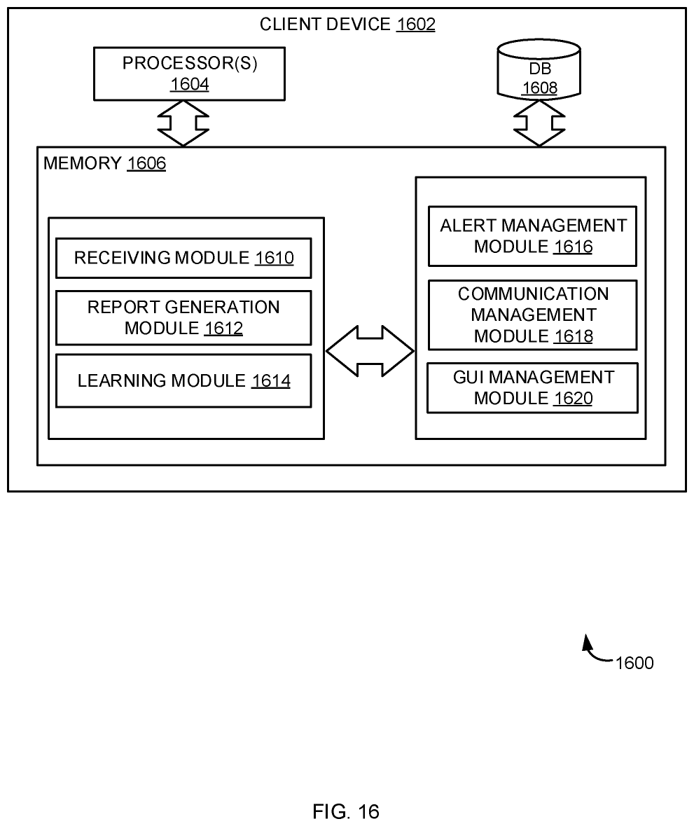

FIG. 16 is a block diagram 1600 of an electronic device/client device 1602 connected to the apparatus for promoting hand hygiene according to the embodiments of the present disclosure. The electronic device may be a server, a virtual machine running in a server, a personal computer, a laptop, a desktop, a smartphone, or a smart wearable. The client device may further include its own processor 1604, memory 1606, and database 1608. Memory 1606 may include a receiving module 1610 for receiving data from the apparatus. A report generation module 1612 processes the received data and generates a compliance report in real-time. The learning module 1614 receives data from the receiving module 1610 and reports from the report generation module 1612 to understand the patterns of hand wash sessions in a location and even identifies user preferences. It may learn how users respond to different types of audio-visual alerts and determine the most efficient type of alert. The suggestions generated by the learning module 1614 may be directly implemented in the apparatus or provided to the user who may choose to implement them manually. The alert management module 1616 receives alerts from the apparatus and provides notifications to the user via the electronic device. Alerts include real-time alerts relating to the usage of the dispenser, battery status, usage level of the hand cleaning agent, and the like. The communication management module 1618 manages communication and data transfer to and from the apparatus. The Graphical User Interface (GUI) management module 1620 manages the user interface of the application that supports the apparatus. Dashboards, notifications, and suggestions are provided to the user via the user interface of the application.

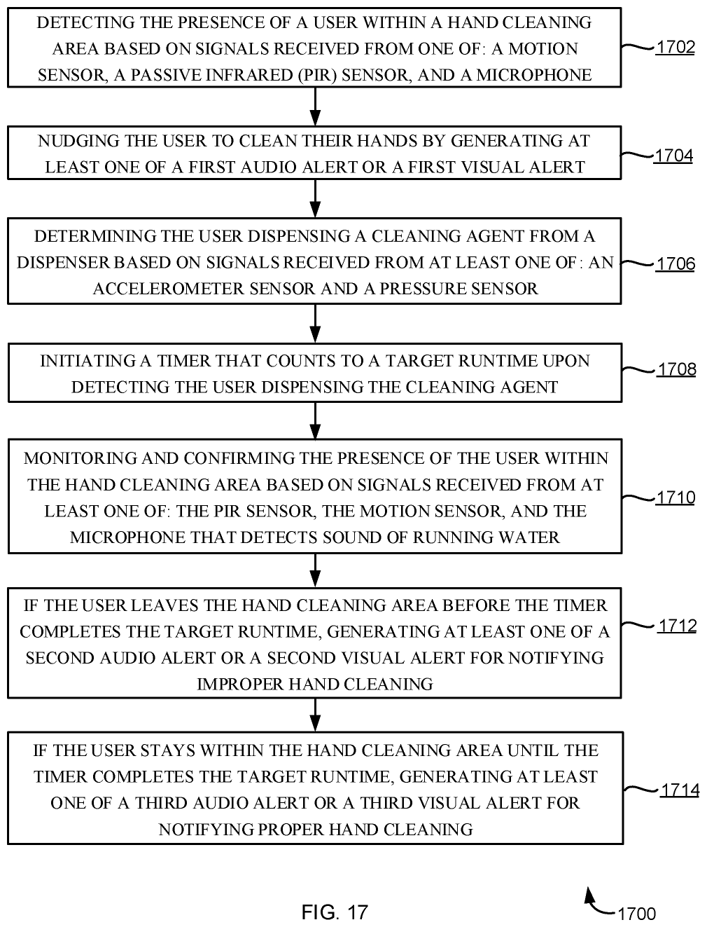

FIG. 17 is a flow diagram 1700 depicting the method for promoting hand hygiene according to the embodiments of the present disclosure. The process depicted in FIG. 17 represents generalized illustrations, and that other processes may be added, or existing processes may be removed, modified, or rearranged without departing from the scope and spirit of the present application. In addition, the processes may represent instructions stored on a computer-readable storage medium that, when executed, may cause a processor to respond, to perform actions, to change states, and/or to make decisions. Alternatively, the processes may represent functions and/or actions, to change states, and/or to make decisions. Alternatively, the processes may represent functions and/or actions performed by functionally equivalent circuits like analog circuits, digital signal processing circuits, application-specific integrated circuits (ASICs), or other hardware components associated with the system. Furthermore, the flow charts are not intended to limit the implementation of the present application, but rather the flowcharts illustrate functional information to design/fabricate circuits, generate programs, or use a combination of hardware and program to perform the illustrated processes.

At 1702, the method performs the step of detecting the presence of a user within a handwashing area based on signals received from at least one of: a passive infrared (PIR) sensor, and a microphone. At 1704, the method performs the step of nudging the user to wash their hands by generating a first audio-visual alert. At 1706, the method performs the step of determining the user dispensing a cleaning agent from a dispenser based on signals received from at least one of: an accelerometer sensor and a pressure sensor. At 1708, the method performs the step of initiating a timer that counts to a target runtime upon detecting the user dispensing the cleaning agent. At 1710, the method performs the step of monitoring and confirming the presence of the user within the handwashing area based on signals received from a combination of: the proximity sensor, the thermal sensor and the microphone that detects sound of running water. At 1712, the method performs the step of generating a second audio-visual alert for notifying improper hand wash if the user leaves the handwashing area before the timer completes the target runtime. At 1714, the method performs the step of generating a third audio-visual alert for notifying proper hand wash if the user stays within the handwashing area until the timer completes the target runtime.

The apparatus described herein may further be configured to transmit usage data of the handwashing dispenser to the client/electronic device in real-time. The usage data may include real-time dispensing information, compliance information, non-compliance information, battery status, and dispenser replacement alert. The usage data received by the electronic device may be processed to generate a hand-hygiene compliance report that provides information including the total number of hand washes detected, the total number of proper hand washes, the total number of improper hand washes, average time spent on all hand washes, average time spent on all improper hand washes, average time spent on all improper hand washes, timestamp of each hand wash, timestamp of all improper hand washes, timestamp of all proper hand washes, the time period during which compliance level was lowest, the time period during which compliance level was highest, user response to different types of audio-visual alerts, and the like. The apparatus may further determine usage level of the hand cleaning agent and creates an alert of the usage level crosses a certain threshold wherein the dispenser needs refill/replacement. A gyrosensor may be used for determining usage level of the hand cleaning agent.

The data received from the apparatus may be analyzed for providing insights into user behavior at different locations where the apparatus is installed. In one example, if the apparatus is installed in different departments of a hospital, an analysis may be provided to rank the user behavior of each department. Time series analysis may also be performed to generate useful results related to compliance at various time periods and locations. The insights generated may be used to improve hand hygiene compliance in areas where the apparatus is used. Furthermore, the apparatus may be allowed to pair with personal electronic devices of users (such as smartphones) to keep track of user behavior related to hand hygiene. For performing this operation, the user may have to provide adequate permissions to the apparatus to track their behavior. In this embodiment, the user via a supporting application may receive personalized recommendations for improving and/or maintaining their hand hygiene.

Various compliance reports, group recommendations, user recommendations, and insights may be used for improving the overall hand hygiene compliance within a facility. The apparatus may be used in homes, offices, factories, hospitals, educational institutions, and the like.

In one example, the apparatus may be attached to a faucet rather than a dispenser. The apparatus disclosed herein may be portable and have a universal fit so that it may be used with faucets of different shapes and sizes available in the market. In one example, the apparatus may comprise of sensors to detect hand movements such as a motion sensor or a passive infrared (PIR) sensor that can register heat to detect hands placed near it. Apart from the above-mentioned, sensors that can detect proximity may also be used for detecting the presence of hands. In other examples, sensors that use a camera or IR camera may also be used to accurately detect the presence of hands and hand movements. Alternatively, a sound detecting sensor may be used to detect the sound of the tap/water flow to monitor the usage time and pattern. Any combination of the above-mentioned sensors may also be used to accurately detect hand movement and the total amount of time spent in hand washing.

In one example, the apparatus disclosed herein may have a video/audio output connected to it and may reward the user after an effective hand wash session. It may be configured to alert users if the handwashing session was not ideal. In another example, it may provide a countdown to encourage users to perform effective hand wash. In another example, it may be paired with a personal electronic device of the user (such as a smartphone) and provide prompts regarding effective and non-effective hand washes performed by the user.

Furthermore, video/IR cameras may also be provided to monitor the hand washing pattern of the individual as well. Apart from counting the total amount of time spent on performing hand wash, these sensors will further monitor if every part of the hands was cleaned properly. It may alert the user for every effective/ineffective hand wash. The apparatus may be configured to send the monitored data to a user's personal device or to a server via Bluetooth or any other related data transfer technology. In one example, the device may even be configured to upload the data to cloud storage so that the data can be accessed from any device. This may help parents monitor the handwashing patterns of their children from a remote location.

In one example, certain areas may be marked as “safe zones” wherein individuals entering these zones must sanitize their hands before entering these zones. In one example, hospital rooms, operation theatres, school buses, and flights are a few examples of regions that may be classified as “safe zones”. In such zones, individuals are expected to sanitize their hands before entering. In such areas, the apparatus disclosed herein may be fixed near the entry doors. In other examples, safe zones could comprise toilets, raw meat processing units, operation theatres, and the like, wherein individuals are expected to sanitize their hands before departing. In such areas, the apparatus may be fixed near the exit doors. In both these cases, the objective is to alert individuals for sanitizing their hands before entering or exiting these zones. In areas such as operation theatres where surgeries are performed on humans/animals, individuals are expected to thoroughly sanitize their hands before entering and before exiting such areas.

In one example, a unidirectional motion sensor may be used to register movement. These sensors may be used to identify the presence of an individual in an area and alerts the individual to use the hand sanitizer before entering the safe zone. The hand sanitizer monitor may keep alerting the individual until it registers the usage of hand sanitizer. A PIR sensor may be used to detect hand movement to successfully register usage of hand sanitizer. If the hand sanitizer is not used as prompted, audio-visual sensors connected to the hand sanitizer monitor may alert one or more users regarding non-compliance.

Furthermore, the motion sensors used herein may have combined features to reduce false alarms. For example, the PIR sensor may be combined with a microwave sensor to reduce false alarms. Each of these sensors operate in different areas of the spectrum, one is passive and one is active thereby reducing chances of false triggers. The advantage of using such dual technology motion sensors is that both of them have to be triggered to register an alarm.

The proposed solution promotes basic hygiene practices such as maintaining clean hands using solutions driven by technology. A combination of sensors and alerting mechanisms is used for promoting hand hygiene among individuals. The proposed solution not only alerts individuals to wash hands but also makes sure that the hand wash performed is effective. Furthermore, sensors are inbuilt and require no further configuration. The devices disclosed herein are plug and play and have a universal fit. They may be compatible with any existing water taps or hand sanitizing modules and dispensers. They are portable and easily attachable/detachable as well.

In one example, LED lights may be provided to display effective and ineffective hand cleaning. Greenlight may depict effective hand cleaning while red lights may depict ineffective cleaning. In another example, pleasant audio may be played as notification for effective hand cleaning while a warning tone may be played for notifying ineffective hand cleaning. In yet another example, a rewarding audio/video message may be played for notifying effecting hand cleaning. Such rewarding messages may encourage kids to clean their hands effectively by following prescribed guidelines. The present disclosure covers all such obvious modifications of light-based, audio-based, and video-based warning and appreciation messages that follow effective/ineffective hand washing/hand sanitizing.

In another example, the apparatus described herein may be retrofitted into existing devices or dispensers. The housing or body of the apparatus may be replaced with the housing of a dispenser or any related apparatus. This way, the technology provided by the present disclosure may be added to existing devices. It may be noted the method disclosed by the present disclosure covers such retrofitting applications.

It may be noted that the above-described examples of the present solution are for the purpose of illustration only. Although the solution has been described in conjunction with a specific embodiment thereof, numerous modifications may be possible without materially departing from the teachings and advantages of the subject matter described herein. Other substitutions, modifications, and changes may be made without departing from the spirit of the present solution. All the features disclosed in this specification (including any accompanying claims, abstract, and drawings), and all of the steps of any method or process so disclosed, may be combined in any combination, except combinations where at least some of such features or steps are mutually exclusive.

The terms “include,” “have,” and variations thereof, as used herein, have the same meaning as the term “comprise” or an appropriate variation thereof. Furthermore, the term “based on”, as used herein, means “based at least in part on.” Thus, a feature that is described as based on some stimulus can be based on the stimulus or a combination of stimuli including the stimulus.

The present description has been shown and described with reference to the foregoing examples. It is understood, however, that other forms, details, and examples can be made without departing from the spirit and scope of the present subject matter that is defined in the following claims.

What is claimed is:

1. An apparatus for promoting effective hand cleaning, the apparatus comprising:
a body removably connected to a hand cleaning agent dispenser, the body comprising:
at least one microcontroller, a speaker, a timer, two or more light-emitting diodes (LEDs), and a plurality of sensors, wherein the plurality of sensors comprise at least one of: a passive infrared (PIR) sensor, an accelerometer sensor, a pressure sensor, a motion sensor, a gyro sensor, and a microphone, and wherein the microcontroller is programmed to:
detect the presence of a user within a range of the apparatus based on signals received from at least one of: the motion sensor, the PIR sensor, and the microphone;
nudge the user to clean their hands by generating at least one of a first audio alert and a first visual alert;
determine the user dispensing a cleaning agent from a dispenser based on signals received from at least one of: the accelerometer sensor, the gyro sensor and the pressure sensor;
initiate the timer that counts to a target runtime upon detecting the user dispensing the cleaning agent;
monitor and confirm the presence of the user within the range of the apparatus area based on the signals received from at least one of: the motion sensor, the PIR sensor and the microphone that detects sound of running water;
generate at least one of a second audio alert and a second visual alert for notifying improper hand cleaning if the user leaves the range of the apparatus before the timer completes the target runtime; and
generate at least one of a third audio alert or a third visual alert for notifying proper hand cleaning if the user stays within the range of the apparatus until the timer completes the target runtime.

2. The apparatus of claim 1, wherein the microcontroller is further programmed to:
transmit usage data of the hand cleaning agent dispenser to an electronic device, wherein the usage data includes: real-time dispensing information, compliance information, non-compliance information, battery status, and dispenser refill alert.

3. The apparatus of claim 2, wherein the usage data received by the electronic device is processed to generate a hand-hygiene compliance report.

4. The method of claim 3, wherein the hand-hygiene compliance report provides information including: total number of hand cleaning detected, total number of proper hand cleaning, total number of improper hand cleaning, average time spent on all hand cleaning, average time spent on all improper hand cleaning, average time spent on all improper hand cleaning, timestamp of each hand cleaning, timestamp of all improper hand cleaning, timestamp of all proper hand cleaning, time period during which compliance level was lowest, and time period during which compliance level was highest.

5. The method of claim 2, wherein the electronic device is a personal computer, a laptop computer, a tablet, a smartphone, or a smart wearable.

6. A method for promoting effective hand cleaning, comprising:
detecting the presence of a user within a hand cleaning area based on signals received from at least one of: a motion sensor, a passive infrared (PIR) sensor, and a microphone;
nudging the user to clean their hands by generating at least one of a first audio alert or a first visual alert;
determining the user dispensing a cleaning agent from a dispenser based on signals received from at least one of: a gyro sensor, an accelerometer sensor and a pressure sensor;
initiating a timer that counts to a target runtime upon detecting the user dispensing the cleaning agent;
monitoring and confirming the presence of the user within the hand cleaning area based on signals received from at least one of: the PIR sensor, the motion sensor, and the microphone that detects sound of running water;
if the user leaves the hand cleaning area before the timer completes the target runtime, generating at least one of a second audio alert or a second visual alert for notifying improper hand cleaning; and
if the user stays within the hand cleaning area until the timer completes the target runtime, generating at least one of a third audio alert or a third visual alert for notifying proper hand cleaning.

7. The method of claim 6, further comprising:
transmitting usage data of the hand cleaning agent dispenser to an electronic device, wherein the usage data includes: real-time dispensing information, compliance information, non-compliance information, and dispenser refill alert.

8. The method of claim 7, wherein the received usage data is used by the electronic device to generate a hand-hygiene compliance report.

9. The method of claim 8, wherein the hand-hygiene compliance report provides information including: total number of hand cleaning detected, total number of proper hand cleaning, total number of improper hand cleaning, average time spent on all hand cleaning, average time spent on all improper hand cleaning, average time spent on all improper hand cleaning, timestamp of each hand cleaning, timestamp of all improper hand cleaning, timestamp of all proper hand cleaning, time period during which compliance level was lowest, and time period during which compliance level was highest.

10. The method of claim 7, wherein the electronic device is a personal computer, a laptop computer, a tablet, a smartphone, or a smart wearable.
